(19) United States
(12) Patent Application Publication
(10) Pub. No.: US 2007/0185573 A1
(43) Pub. Date: Aug. 9, 2007

(54) Load modulation communication circuit and visual restoration aiding device provided with the same

(75) Inventor: Eiji Yonezawa, Okazaki-shi (JP)

Correspondence Address: OLIFF & BERRIDGE, PLC, P.O. BOX 19928, ALEXANDRIA, VA, 22320, US

(73) Assignee: NIDEK CO., LTD., GAMAGORI-SHI (JP)

(21) Appl. No.: 11/700,868

(22) Filed: Feb. 1, 2007

(30) Foreign Application Priority Data
Feb. 6, 2006 (JP) 2006-028963

(51) Int. Cl. A61F 2/14; A61N 1/00

(52) U.S. Cl. 623/4.1; 607/54

(57) ABSTRACT

A load modulation communication circuit connected to a secondary coil arranged facing a primary coil, comprises: a center tap connected to the secondary coil; a switch unit connected at its one end to the center tap via a resistor and at the other end to approximately ground; and a control circuit which is connected to the switch unit to control an on/off action of the switch unit and also connected to both ends of the secondary coil or one end of the secondary coil and the center tap to control communications through the secondary coil by controlling the on/off action of the switch unit; wherein when the switch unit is off, the secondary coil obtains electric power and receives the information from the primary coil, while when the switch unit is on, the center tap is connected to approximately ground via the resistor and thus a load on the secondary coil is increased, thereby causing the secondary coil to transmit information to the primary coil.

### BACKGROUND OF THE INVENTION

1. Field of the Invention

The present invention relates to a load modulation communication circuit used for a RFID (radio frequency identification) technique and a visual restoration aiding device provided with the load modulation communication circuit.

2. Description of Related Art

There is known a RFID technique that performs contactless communications of information and electric power (see e.g. U.S. Pat. No. 6,837,438 (JP-A-2000-137779)). This RFID technique is called wireless IC tag and of wide applicability. As the applicability of such wireless IC tag is expanded, it is required that a receiving device is low in power consumption and compact in size. Therefore a load modulation communication circuit widely used in the receiving device particularly needs to be low in power consumption and compact in size.

As a blindness cure apparatus, recently, there is proposed a visual restoration aiding device arranged to apply electrical stimulation from electrodes to cells generating vision to serve as part of a lost visual function. This visual restoration aiding device has an internal (intracorporeal) device which is to be placed (embedded) in a body and comprises electrodes and a control part including an integrated circuit which controls the electrodes. Using the RFID technique, such visual restoration aiding device can supply (transmit) information and electric power from outside of the body to the internal device. It is preferable that the supplied electric power is consumed effectively in the internal device. Further, because the internal device is to be placed in the body of a patient, it is preferably designed as compactly and simply as possible.

### BRIEF SUMMARY OF THE INVENTION

The present invention has been made in view of the above circumstances and has an object to provide a load modulation communication circuit with a simple configuration to be driven with low power consumption and further provide a visual restoration aiding device provided with such load modulation communication circuit.

Additional objects and advantages of the invention will be set forth in part in the description which follows and in part will be obvious from the description, or may be learned by practice of the invention. The objects and advantages of the invention may be realized and attained by means of the instrumentalities and combinations particularly pointed out in the appended claims.

To achieve the purpose of the invention, there is provided a load modulation communication circuit connected to a secondary coil arranged facing a primary coil, comprising: a center tap connected to the secondary coil; a switch unit connected at its one end to the center tap via a resistor and at the other end to approximately ground; and a control circuit which is connected to the switch unit to control an on/off action of the switch unit and also connected to both ends of the secondary coil or one end of the secondary coil and the center tap to control communications through the secondary coil by controlling the on/off action of the switch unit; wherein when the switch unit is off, the secondary coil obtains electric power and receives the information from the primary coil, while when the switch unit is on, the center tap is connected to approximately ground via the resistor and thus a load on the secondary coil is increased, thereby causing the secondary coil to transmit information to the primary coil.

According to another aspect, the present invention provides a vision restoration aiding device comprising, a primary coil placed outside a body; a secondary coil placed inside the body and arranged facing the primary coil; a load modulation communication circuit connected to the secondary coil; a plurality of electrodes placed inside the body; and a control part which converts information transmitted from the primary coil and received by the secondary coil to an electrical stimulation pulse signal and outputs the signal from the electrodes to cells constituting a retina; wherein the load modulation communication circuit comprises: a center tap connected to the secondary coil; a switch unit connected at its one end to the center tap via a resistor and at the other end to approximately ground; and a control circuit which is connected to the switch unit to control an on/off action of the switch unit and also connected to both ends of the secondary coil or one end of the secondary coil and the center tap to control communications through the secondary coil by controlling the on/off action of the switch unit; wherein when the switch unit is off, the secondary coil obtains electric power and receives information from the primary coil, while when the switch unit is on, the center tap is connected to approximately ground via the resistor and thus a load on the secondary coil is increased, thereby causing the secondary coil to transmit information to the primary coil.

### BRIEF DESCRIPTION OF THE DRAWINGS

The accompanying drawings, which are incorporated in and constitute a part of this specification illustrate an embodiment of the invention and, together with the description, serve to explain the objects, advantages and principles of the invention.

In the drawings,

FIG. 1 is a schematic structural view of an external device of a visual restoration aiding device in a preferred embodiment of the present invention;

FIG. 2 is a schematic structural view of an internal device of the visual restoration aiding device;

FIG. 3 is a schematic circuit diagram of a load modulation communication circuit in the preferred embodiment of the present invention;

FIG. 4 is a schematic block diagram of a control system of the visual restoration aiding device; and

FIG. 5 is a view showing a state where a stimulation unit of the internal device is placed in a body of a patient.

### DETAILED DESCRIPTION OF THE PREFERRED EMBODIMENTS

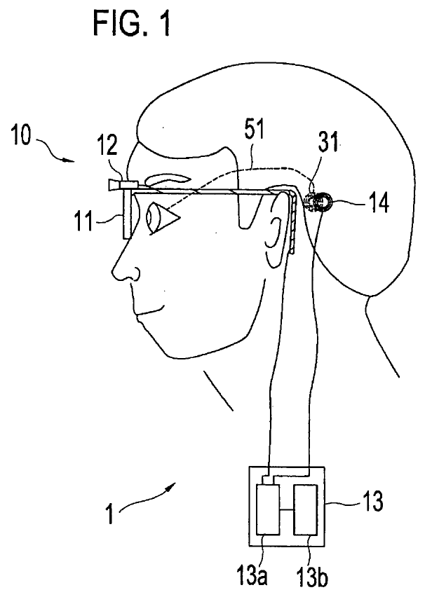

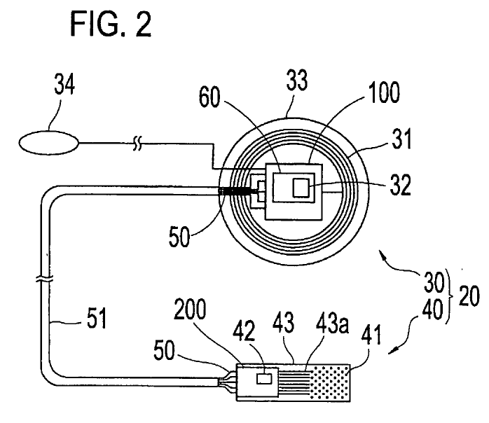

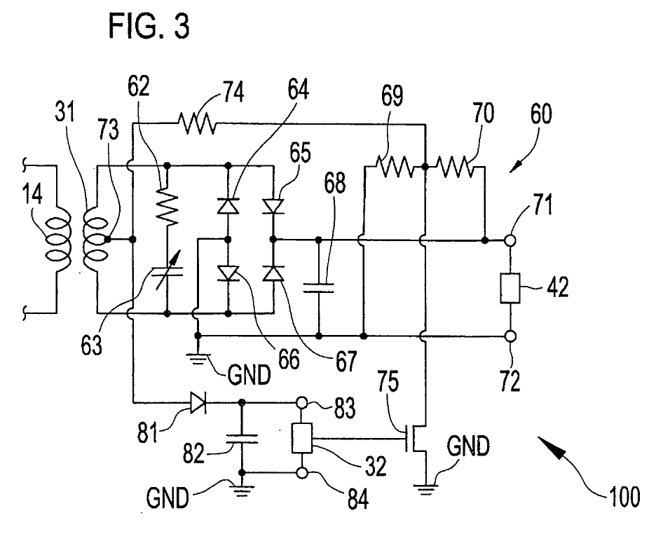

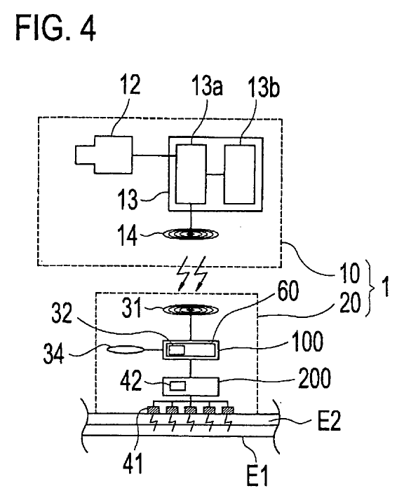

A detailed description of a preferred embodiment of the present invention will now be given referring to the accompanying drawings. The following explanation will be made on a visual restoration aiding device exemplified as one embodiment using a load modulation communication circuit. FIG. 1 is a schematic structural view of an external device of a visual restoration aiding device in the present embodiment of the present invention. FIG. 2 is a schematic structural view of an internal device of the visual restoration aiding device. FIG. 3 is a schematic circuit diagram of a load modulation communication circuit in the preferred embodiment of the present invention. FIG. 4 is a schematic block diagram of a control system of the visual restoration aiding device.

A visual restoration aiding device 1 includes an external (extracorporeal) device 10 for photographing external world (surrounding images) and an internal (intracorporeal) device 20 for inducing restoration of vision by applying electrical stimulation to cells constituting a retina. The external device 10 has a visor 11 which a patient wears, a photographing unit 12 such as a CCD camera attached to the visor 11, an external unit 13, and a primary coil (a transmitting/receiving unit) 14. The visor 11 has a spectacles appearance, which the patient wears in front of his/her eyes during use. The photographing unit 12 is attached to the front of the visor 11 to photograph an object to be recognized by the patient.

The external unit 13 includes a processing unit (a data modulation unit) 13a provided with a calculation and processing circuit, and a power source unit (a battery) 13b for supply of electric power to the device 1 (the external device 10 and the internal device 20). The processing unit 13a processes image data captured by the photographing unit 12 and converts it to data for electrical stimulation pulse signals, the data including the intensity of the electrical stimulation pulse signals, stimulation positions, and others. The primary coil 14 transmits, in the form of electromagnetic waves, the data for electrical stimulation pulse signals converted by the processing unit 13a and the electric power from the power source unit 13b via the processing unit 13a to the internal device 20. The primary coil 14 is provided with a centrally-located magnet which is used for fixing the position relative to a secondary coil 31 mentioned below.

The internal device 20 includes a communication unit 30 which receives the information such as the data for electrical stimulation pulse signals and the electric power transmitted from the external device 10 and a stimulation unit 40 which applies electrical stimulation to cells constituting a retina.

The communication unit 30 serves to receive the information such as the data for electrical stimulation pulse signals and the electric power transmitted from the external device 10 and also to transmit predetermined information to the external device 10. The communication unit 30 includes the secondary coil (a transmitting/receiving unit) 31 and a control part 100 provided with a load modulation communication circuit 60 including a control circuit 32. The control part 100 serves to divide the received signal into the information such as the data for electrical stimulation pulse signals and the electric power and also to convert the data for electrical stimulation pulse signals to the electrical stimulation pulse signals.

The secondary coil 31 and the control part 100 of the communication unit 30 are formed on a substrate 33. At the center of the secondary coil 31, a magnet is attached to be used for fixing the position relative to the primary coil 14.

The stimulation unit 40 includes a plurality of electrodes 41 which output the electrical stimulation pulse signals and a control part 200 provided with a control circuit 42. Each electrode 41 is connected to the control part 200 (the control circuit 42). The control part 200 serves to distribute the electrical stimulation pulse signals to the electrodes 41 in accordance with the signal from the control part 100 and output the signals from the electrodes 41 to the cells constituting the retina.

The electrodes 41 and the control part 200 of the stimulation unit 40 are formed on a flexible substrate 43. Each electrode 41 is connected to the control part 200 (the control circuit 42) with conductive wires 43a.

The communication unit 30 and the stimulation unit 40 are coupled to each other with a plurality of conductive wires 50 bundled together by a tube 51.

The control part 100 (the load modulation communication circuit 60) of the communication unit 30 will be explained below referring to FIG. 3.

The primary coil 14 and the secondary coil 31 are arranged facing each other to cause information communications and power supply owing to electromagnetic induction occurring therebetween. It is to be noted that the electric power obtained at the secondary coil 31 is an alternating voltage. Numeral 62 denotes a resistor and numeral 63 denotes a variable condenser (a variable capacitor). The secondary coil 31 and the condenser 63 constitute a resonance circuit, which extracts a signal of a specific frequency. Owing to the condenser 63 being variable, a resonance frequency can be controlled.

Diodes 64 to 67 convert the alternating voltage to a direct voltage. A condenser (a capacitor) 68 stores and discharges the direct voltage. These diodes 64 to 67 and condenser 68 constitute a full-wave rectifying circuit (rectifying means). In the present embodiment, the four diodes 64 to 67 form a diode bridge and they configure the rectifying circuit in combination with the condenser 68. However, another configuration may be adopted; for example, a MOSFET (metal oxide semiconductor field effect transistor) may be used.

Resistors 69 and 70 serve to reduce a potential at a center tap 73 to be mentioned later to about half of a sum of a potential of an output line (a power output terminal 71) and an approximately ground potential. Accordingly, respective resistance values of the resistors 69 and 70 are set to be almost equal.

Numerals 71 and 72 indicate power output terminals; one terminal 71 is at a positive potential and the other terminal 72 is at an approximate ground potential. These power output terminals 71 and 72 are connected to the control circuit 42 and output the direct voltage to the same

The center tap 73 is connected to an almost halfway point of the secondary coil 31, namely, a point corresponding to about half the total number of turns of the secondary coil 31. Numeral 74 denotes a resistor. Numeral 75 denotes a FET (Field Effect Transistor) whose drain is connected to the resistor 74, source is connected to approximately ground, and gate is connected to the control circuit 32.

A diode 81 is connected at its anode to the center tap 73. A condenser (a capacitor) 82 is connected at its one end to the cathode of the diode 81 and at the other end to approximate ground. The diode 81 converts the alternating voltage to the direct voltage. The condenser 82 stores and discharges the direct voltage. These diode 81 and condenser 82 constitute a half-wave rectifying circuit (rectifying means).

Numerals 83 and 84 denote power output terminals; one terminal 83 is at a positive potential and the other terminal 84 is at the approximate ground potential. These power output terminals 83 and 84 are connected to the control circuit 32 and they output the direct voltage to the same.

When the FET 75 is switched on in response to a command signal from the control circuit 32, the potential at one end of the resistor 74 drops to the approximate ground potential. This forms a current pathway from the center tap 73 to the resistor 74. Further, when the FET 75 is switched off in response to a command signal from the control circuit 32, the source and the drain in the FET 75 are brought out of conduction. When the FET 75 is off, power loss in the FET 75 and the resistor 74 will be reduced.

The FET 75 may be either an n-type MOSFET or a p-type MOSFET. If the FET 75 is the n-type MOSFET, a positive voltage is applied to its gate to switch on the FET 75. If it is the p-type MOSFET, alternatively, a negative voltage is applied to its gate to switch on the FET 75.

In the case where the information is to be transmitted from the secondary coil 31, the FET 75 is switched on. This allows the center tap 73 to be connected to approximately ground via the resistor 74, so that the load on the secondary coil 31 increases by a degree caused by the resistor 74. Since the primary coil 14 and the secondary coil 31 are electromagnetically coupled to each other, voltage amplitude in the primary coil 14 will decreases as the load on the secondary coil 31 increases. This change in voltage amplitude in the primary coil 14 is sensed by the external unit 13 (the processing unit 13a), and thus operating state of the internal device 20 is detected. This is determined as representing that the information has been transmitted from the secondary coil 31 to the primary coil 14. Such communications are repeated at regular intervals (e.g., every tens to hundreds milliseconds)

While the control circuit 32 is operating for applying the electrical stimulation to the cells constituting the retina (i.e., outputting the electrical stimulation pulse signals), the internal device 20 (the control part 100) periodically transmits the information that the internal device 20 is normally operating to the external device 10 (the external device 10 periodically receives that information). Specifically, the operating state of the internal device 20 is periodically informed from the internal device 20 to the external device 10. If not obtaining the operating state of the internal device 20 even after a lapse of a predetermined time, which is regarded as representing that either or both of the internal device 20 and the external device 10 have failures, the external device 10 notifies thereof with a buzzer, light, or the like not shown.

A resistance value of the resistor 74 is set to a large value in a range permitting communications but causing no interruption in power supply.

As mentioned above, load modulation means is made up of the center tap 73, resistance 74, and FET 75, so that the load modulation communication circuit 60 can be formed with a simple structure. Since the center tap 73 is held stably at a midpoint potential and thus no high frequency voltage appears, no current will flow in the resistor 74 even through the capacitance of the FET 75 exists, resulting in a reduction in power loss.

When the FET 75 is off, the secondary coil 31 obtains voltage from the primary coil 14. The obtained voltage is rectified and smoothed by the rectifying circuit made up of the diodes 64 to 67 and the condenser 68, and then supplied to the control circuit 42 via the power output terminals 71 and 72.

With a combination of the center tap 73, the diode 81, and the condenser 82, further, a half voltage of the voltage the secondary coil 31 obtains is supplied to the control circuit 32 via the power output terminals 83 and 84.

The control circuit 32 and the control circuit 42 are supplied with different voltages as above because they require different voltages. In the present embodiment, the control circuit 32 is a semiconductor circuit that is activated by a voltage of 3.3V, while the control circuit 42 is configured to need a high voltage for applying the electrical stimulation to the cells constituting the retina; that is, a voltage of 10V in the present embodiment. According to the present invention, using the center tap 73, the control circuit 32 is supplied with a voltage of 5V when the control circuit 42 is supplied with a voltage of 10V. The control circuit 32 lowers the supplied voltage of 5V to a voltage of 3.3V in use. In this way, appropriate electric power can be supplied to each element of the internal device 20, thereby reducing power loss in the internal device 20 and improving use efficiency of electric power.

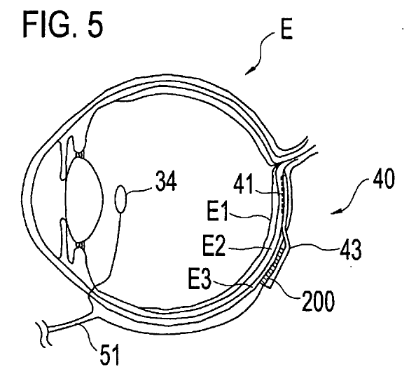

The internal device 20 having the above structure is disposed in a predetermined position inside the patient's body. FIG. 5 is a view showing a state where the stimulation unit 40 of the internal device 20 is placed in the patient's body. A portion of the substrate 43 provided with the electrodes 41 is placed between a choroid E2 and a sclera E3 by bringing the electrode 41 into contact with the choroid E2. Another portion of the substrate 43 provided with the control part 200 is placed outside the sclera E3.

An indifferent electrode 34 is implanted in the patient's eye E (in a vitreous body), at a site closer than the center to an anterior segment of the eye E. Accordingly, a retina E1 is positioned between the electrodes 41 and the indifferent electrode 34. The electrical stimulation pulse signals from the electrodes 41 are thus allowed to efficiently pass through the retina E1.

The secondary coil 31 of the communication unit 30 of the internal device 20 is placed in a predetermined position in the body where it can receive the signal (the information such as the data for electrical stimulation pulse signals and the electric power) from the primary coil 14 of the external device 10. For example, as shown in FIG. 1, the communication unit 30 (the secondary coil 31) is placed under the skin of a temporal region of the patient and the primary coil 14 is located on the opposite side of the skin to the communication unit 30 (the secondary coil 31). Since the primary coil 14 and the secondary coil 31 are attached with magnets respectively, they magnetically attract each other, thereby maintaining the primary coil 14 on the skin of the temporal region.

The tube 51 composed of the bundled wires 50 is disposed in such a manner as to extend under the skin of the temporal region from the control part 100 of the communication unit 30 to the eye E and pass the inside of an upper eyelid into an orbit. The tube 51 inserted in the orbit passes the outside of the sclera E3 and is connected to the control part 200 of the stimulation unit 40 as shown in FIG. 5.

In the present embodiment, the stimulation unit 40 (the internal device 20) is placed between the choroids E2 and the sclera E3 and on the outside of the sclera E3, but it is not limited thereto. Specifically, the stimulation unit 40 has only to be placed in such a position as to appropriately provide the electrical stimulation from the electrodes to the cells constituting the retina El. For instance, the stimulation unit 40 (the internal device 20) may be placed between the retina E1 and the choroid E2 and on the inside of the retina E1.

It should be noted that the control circuits 32 and 42 may be configured integrally instead of being arranged separately.

The internal device 20 may receive the information such as the data for electrical stimulation pulse signals and the electric power from the external device 10 either only when the FET 75 is off or when the FET 75 is both on and off, i.e., at any time.

In the aforementioned load modulation communication circuit 60, the center tap 73 is connected to the halfway point of the secondary coil 31. However, the center tap 73 may be connected to any point determined by dividing the total length of the secondary coil 31 at a predetermined ratio; for example, 4:6, 3:7, etc.

While the presently preferred embodiment of the present invention has been shown and described, it is to be understood that this disclosure is for the purpose of illustration and that various changes and modifications may be made without departing from the scope of the invention as set forth in the appended claims.

What is claimed is:

1. A load modulation communication circuit connected to a secondary coil arranged facing a primary coil, comprising:
a center tap connected to the secondary coil;
a switch unit connected at its one end to the center tap via a resistor and at the other end to approximately ground; and
a control circuit which is connected to the switch unit to control an on/off action of the switch unit and also connected to both ends of the secondary coil or one end of the secondary coil and the center tap to control communications through the secondary coil by controlling the on/off action of the switch unit;
wherein when the switch unit is off, the secondary coil obtains electric power and receives the information from the primary coil, while when the switch unit is on, the center tap is connected to approximately ground via the resistor and thus a load on the secondary coil is increased, thereby causing the secondary coil to transmit information to the primary coil.

2. The load modulation communication circuit according to claim 1, wherein the center tap is connected to a halfway point of the secondary coil.

3. The load modulation communication circuit according to claim 1, wherein the switch unit is a field effect transistor.

4. A vision restoration aiding device comprising:
a primary coil placed outside a body;
a secondary coil placed inside the body and arranged facing the primary coil;
a load modulation communication circuit connected to the secondary coil;
a plurality of electrodes placed inside the body; and
a control part which converts information transmitted from the primary coil and received by the secondary coil to an electrical stimulation pulse signal and outputs the signal from the electrodes to cells constituting a retina;
wherein the load modulation communication circuit comprises:
a center tap connected to the secondary coil;
a switch unit connected at its one end to the center tap via a resistor and at the other end to approximately ground; and
a control circuit which is connected to the switch unit to control an on/off action of the switch unit and also connected to both ends of the secondary coil or one end of the secondary coil and the center tap to control communications through the secondary coil by controlling the on/off action of the switch unit;
wherein when the switch unit is off, the secondary coil obtains electric power and receives information from the primary coil, while when the switch unit is on, the center tap is connected to approximately ground via the resistor and thus a load on the secondary coil is increased, thereby causing the secondary coil to transmit information to the primary coil.

5. The vision restoration aiding device according to claim 4, wherein the center tap is connected to a halfway point of the secondary coil.

6. The vision restoration aiding device according to claim 4, wherein the switch unit is a field effect transistor.
